(19) United States
(12) Patent Application Publication
(10) Pub. No.: US 2018/0049915 A1
(43) Pub. Date: Feb. 22, 2018

(54) FORCED AIR TEMPERATURE REGULATING PAD WITH CHEST WARMING FEATURE

(71) Applicant: BCG Medical, LLC, San Diego, CA (US)

(72) Inventor: Brandon Cuongquoc Giap, San Diego, CA (US)

(21) Appl. No.: 15/786,150

(22) Filed: Oct. 17, 2017

Related U.S. Application Data
(63) Continuation-in-part of application No. 14/574,722, filed on Dec. 18, 2014.
(60) Provisional application No. 61/918,668, filed on Dec. 20, 2013.
(60) Provisional application No. 62/409,794, filed on Oct. 18, 2016.

(51) Int. Cl. A61F 7/08

(52) U.S. Cl. CPC A61F 7/08; A61F 2007/006

(57) ABSTRACT

A temperature regulating pad has an air-permeable top layer, a perforated middle layer, and a bottom layer. The perforated middle layer is located between the top layer and the bottom layer. The top layer and the perforated middle layer delimit an upper airspace and the perforated middle layer and the bottom layer delimit a lower airspace. A slot is disposed through the pad to create a flap that can fold over onto the top of the patient. Forced air is received into the lower airspace and flows from the lower airspace into the upper airspace through the perforated middle layer, exiting the air-permeable top layer into an external airspace surrounding the patient. With the patient resting on the pad and the foldable flap deployed, warm air flows directly from underneath and above to regulate the temperature of the patient.

### CROSS REFERENCE TO RELATED APPLICATIONS

This application is a continuation-in-part of U.S. patent application Ser. No. 14/574,722 filed on Dec. 18, 2014, which application claims priority to U.S. Provisional Application No. 61/918,668 filed on Dec. 20, 2013. This application also claims priority to U.S. Provisional Application No. 62/409,794 filed Oct. 18, 2016.

### BACKGROUND

Hypothermia is a recognized common occurrence for patients during surgery. Patients who develop hypothermia are at a greater risk for complications, including a greater chance of heart problems, high rates of infection, increase blood loss and prolonged recovery. To counter this, medical personnel may cover the patient with blankets. Blankets are typically bulky, frequently unravel, and may fall off the patient during pre-surgery, surgery, post-surgery, or transport. Additionally, blankets may pose a safety risk and may interfere with the doctor or other personnel's ability to care for the patient. The use of forced warm air blankets or pads is known in the art; however, there are several downfalls. For example, these types of devices typically infuse predetermined high-temperature air through a bladder or pad to maintain the patient's body temperature in the normal range. The technology often utilizes a double layer or bladder type blanket made of a thin air proof bottom material in a perforated top layer. Warm air is forced into the bladder such that warm air is allowed to escape the perforated top layer and come in contact with the patient's skin area that is exposed to the blanket or pad. The shortcomings of this technology is that the tiny air jets coming out from the double layer blanket or pad can generate are relatively turbulent and, therefore, can cause the excitement of dust contained in or adjacent to the blanket or pad. This turbulent and potentially dusty air can compromise the otherwise engineered and sterile airflow created in the operating room. There are concerns, though not proven, of increased infection associated with the use of forced warm air technology using the traditional double layer or bladder technology as a result of the dust problem.

In addition, traditional under-body temperature regulating pads do not have the ability to directly warm (or cool) the portion of the patient's body that is not in contact with the underlying pad. For example, if the patient is laying supine on his or her back, the anterior of the body is not in direct contact with the underlying pad and, therefore, the chest, torso, legs and the like can only be warmed (or cooled) from the pad through indirect airflow.

Accordingly, the present disclosure is directed to a temperature regulating pad or blanket that has reduced turbulence of air coming exiting the blanket or pad to the patient. The temperature regulating pad or blanket is configured to generate an even air distribution (whether warm or cool) through the blanket toward the patient. Additionally, the temperature regulating pad or blanket includes a slot such that the pad can be folded over on top of the patient's upper body or lower body regions to provide additional warming capability directly to the exposed surface of the body.

### BRIEF DESCRIPTION OF THE DRAWINGS

FIG. 1 is a perspective view of one embodiment of the temperature regulating pad.

FIG. 2 is a cross-sectional view of one embodiment of the temperature regulating pad.

FIG. 3 is a top view of the middle layer of the temperature regulating pad, showing the construction thereof

FIG. 4 is a perspective view of one embodiment of the temperature regulating pad in an inflated state with forced warm air passing therethrough.

FIG. 5 is a cross-sectional view of one embodiment of the temperature regulating pad in an inflated state with forced warm air passing therethrough.

FIG. 6 is a perspective view of another embodiment of the temperature regulating pad with the chest flap feature.

FIG. 7A is a perspective view demonstrating a patient disposed on the temperature regulating pad shown in FIG. 6 with the chest flap feature un-deployed.

FIG. 7B is a perspective view demonstrating a patient disposed on the temperature regulating pad shown in FIG. 6 with the chest flap feature deployed.

FIG. 8A is a view of one embodiment of the temperature regulating pad in an inflated state in use with a patient on surgical table, demonstrating the flow of warm air.

FIG. 8B is another view of one embodiment of the temperature regulating pad in an inflated state in use with a patient on surgical table, demonstrating the flow of warm air.

FIG. 8C is yet another view of one embodiment of the temperature regulating pad in an inflated state in use with a patient on surgical table, demonstrating the flow of warm air.

FIG. 9 is a perspective view of one embodiment of the temperature regulating pad in an inflated state in use with a patient on surgical table and attached to a warm air source.

### DETAILED DESCRIPTION

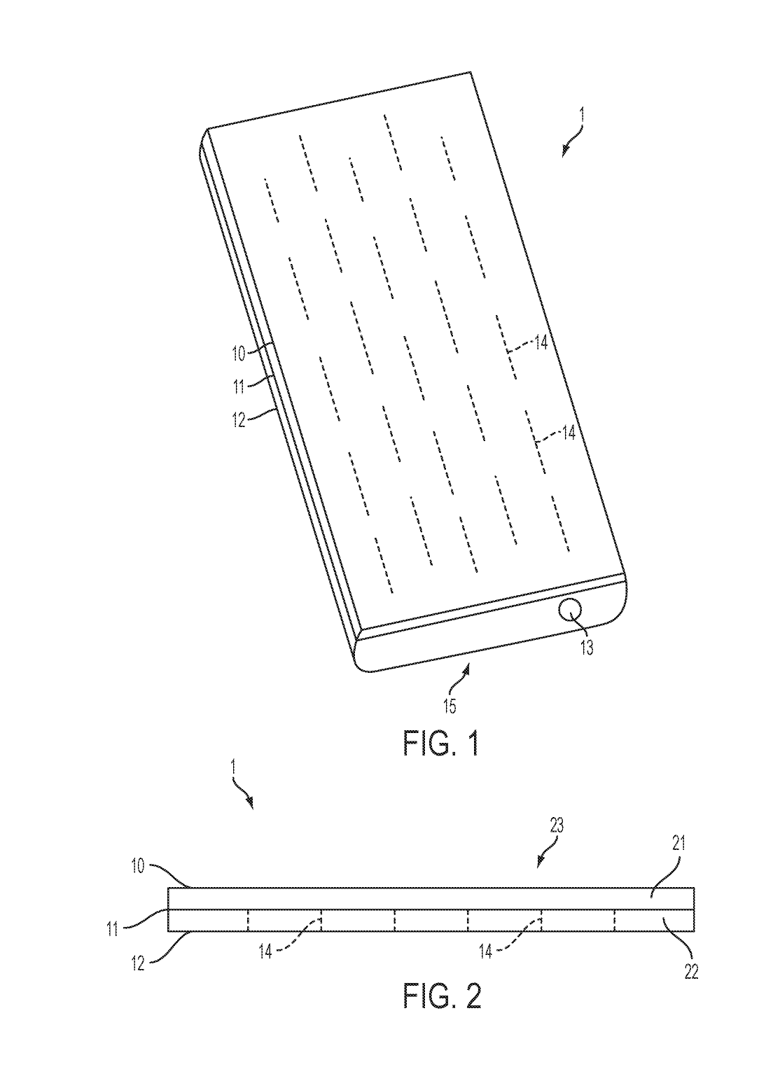

Shown in FIG. 1 is temperature regulating pad 1 generally configured as a planar element having a top layer 10, a middle layer 11, and a bottom layer 12. Temperature regulating pad 1 is shown at least partially transparent in FIG. 1 whereby a plurality of seal lines 14 attaching middle layer 11 to bottom layer 12 can be seen. Air inlet 13 may also be provided, in some embodiments, along the bottom edge 15 of the temperature regulating pad 1. Other locations for the air inlet 13 are suitable, for example on a side edge or the top edge of the pad 1 or other such location that will permit unobstructed flow of forced air. The temperature regulating pad 1 may be configured as a standalone device to be placed on a stretcher, surgical table, bed, or other support surface. Alternatively, the temperature regulating pad 1 may be configured to be inserted into a sheet or pocket provided, for example, in the patient positioning device described in Applicant's patient positioning device invention described in U.S. application Ser. No. 14/340,611, U.S. application Ser. No. 13/359,734 and U.S. Pat. No. 8,661,580.

FIG. 2 shows a cross-section of the temperature regulating pad 1. As shown, middle layer 11 is disposed between bottom layer 12 and top layer 10. This construction divides the temperature regulating pad 1 into an upper airspace 21 delimited by the top layer 10 and middle layer 11, and a lower airspace 22 delimited by the middle layer 11 in the bottom layer 12. An external airspace 23 is located about and above top layer 10 and generally defines the area in which forced air is communicated to a patient resting on the temperature regulating pad 1. In some embodiments, a plurality of seal lines 14 attach the middle layer 11 to the bottom layer 12 at spaced apart intervals across the width of the temperature regulating pad 1. Referring back to FIG. 1, seal lines 14 are also disposed along the length of the pad 1 in spaced-apart fashion. In some embodiments, seal lines 14 are disposed in a matrix formation with respect to the surface of the temperature regulating pad 1. Air inlet 13 shown in FIG. 1 is located to be in flow communication with and introduce air into the lower airspace 22 and, therefore, is disposed between the middle layer 11 and bottom layer 12, in some cases along the bottom edge 15 thereof although other positions around the exterior perimeter of the temperature regulating pad 1 may be equally suitable.

In some embodiments, bottom layer 12 comprises a low friction nylon material and is substantially impermeable to air and is waterproof In some embodiments, the middle layer 11 comprises a thin plastic sheet of which at least a portion of is perforated to include a plurality of apertures 111. In some embodiments the apertures 111 are dispersed evenly along at least a portion of the surface area of the middle layer 11. In some embodiments, the top layer 10 comprises an air-permeable, breathable but waterproof material, such as polypropylene, which will allow air to pass through from the upper airspace 21 to the external airspace 23 in order to warn a patient disposed on the temperature regulating pad 1.

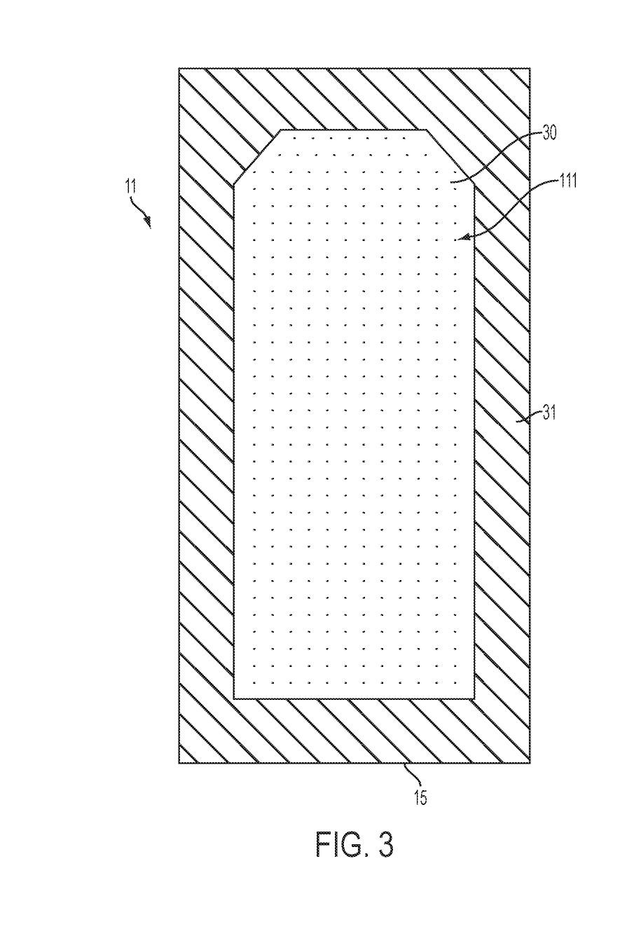

FIG. 3 is a top view of an embodiment of the middle layer 11 showing the construction thereof In some embodiments, middle layer 11 includes a perforated center section 30 comprising the aforementioned perforated surface of the middle layer 11, which is surrounded by an impermeable border 31. As shown, the perforated center section 30 includes the plurality of apertures 111 disposed therethrough. The impermeable border 31 reinforces the construction of the temperature regulating pad 1 and also assures that air is forced up through the perforated center section 30 and does not get trapped at the terminal sides of the temperature regulating pad 1. The width of the impermeable border 31 can vary depending on the desired construction; however, it should be sufficiently wide such as to generally cause the majority of introduced air to pass through the perforated center section 30, rather than to get trapped at the edges of the temperature regulating pad 1.

In some embodiments, the impermeable border 31 has a substantial transverse thickness relative to the overall width and length of the pad 1 such that the border 31 comprises a substantial portion of the surface area of the middle layer 11. In some embodiments, the thickness is substantially wider than an ordinary seal line. In some embodiments, the width of the perforated center section is approximately half of the entire width of the pad, with the impermeable border comprising the remaining width, at least with respect to the middle layer 11.

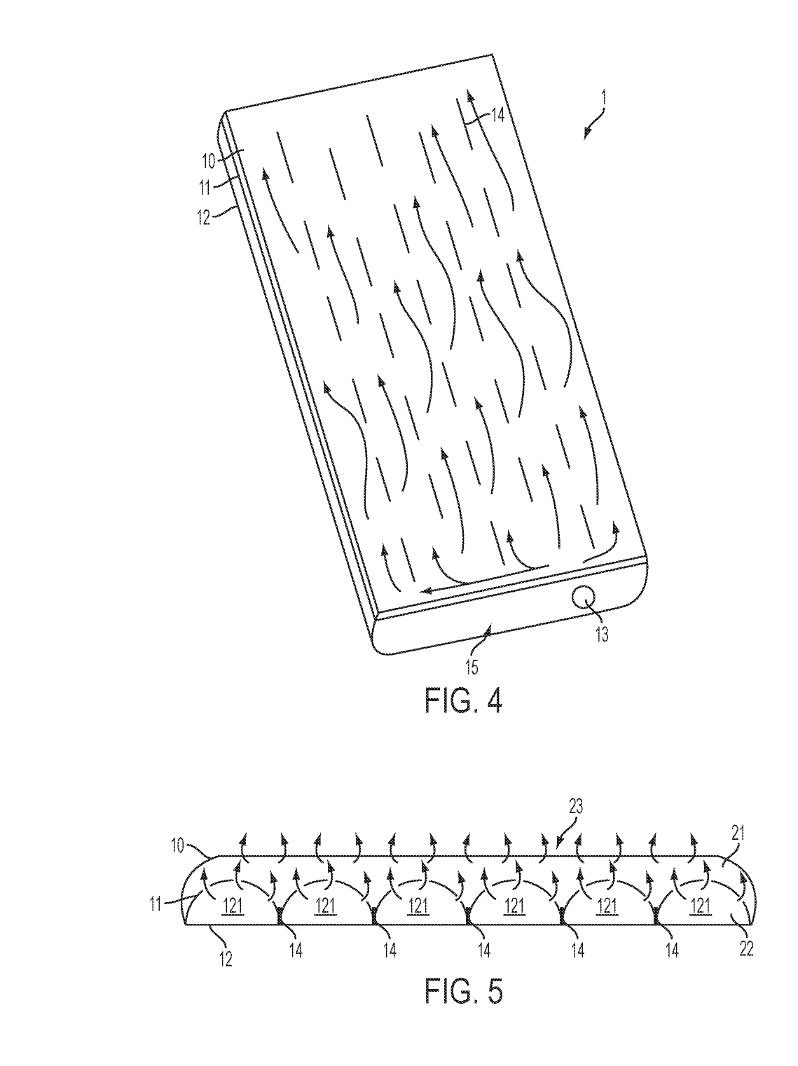

In other embodiments, the width of the perforated center section is approximately two-thirds of the width of the pad, with the impermeable border comprising the remaining width, at least with respect to the middle layer 11. As noted, the relatively wide impermeable border 31 assures that air is forced through the center section 30 to the body mass of the patient and prevents air from being trapped, wasted, or lost at the terminal sides of the pad 1 where such air would otherwise provide no appreciable airflow at or toward the patient resting thereon. FIGS. 4 and 5 depict the temperature regulating pad 1 in an inflated or action state.

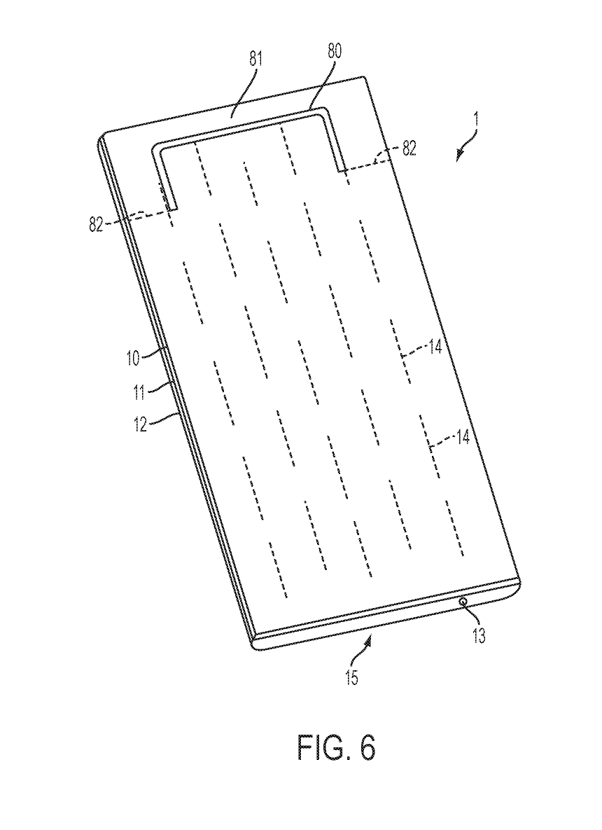

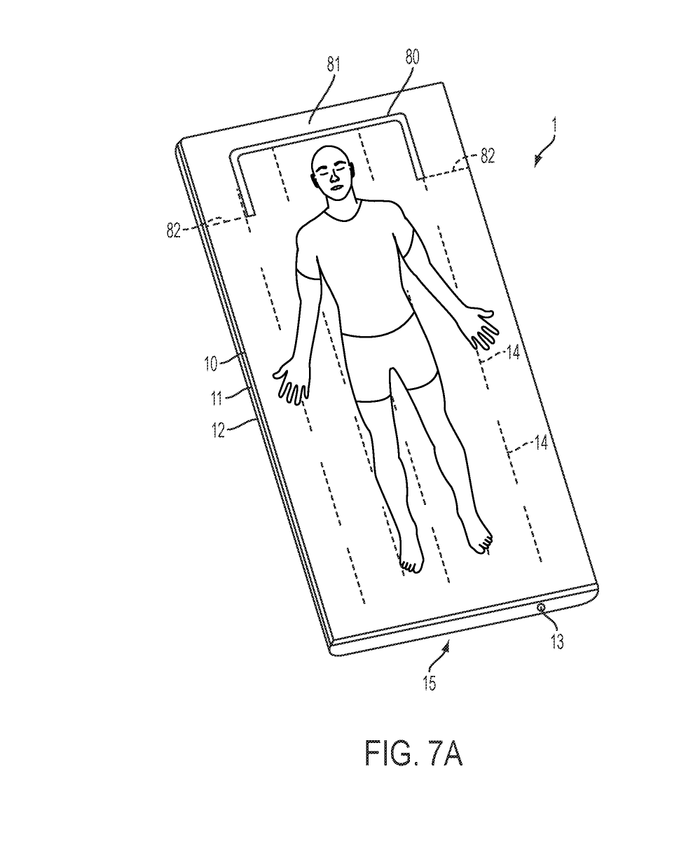

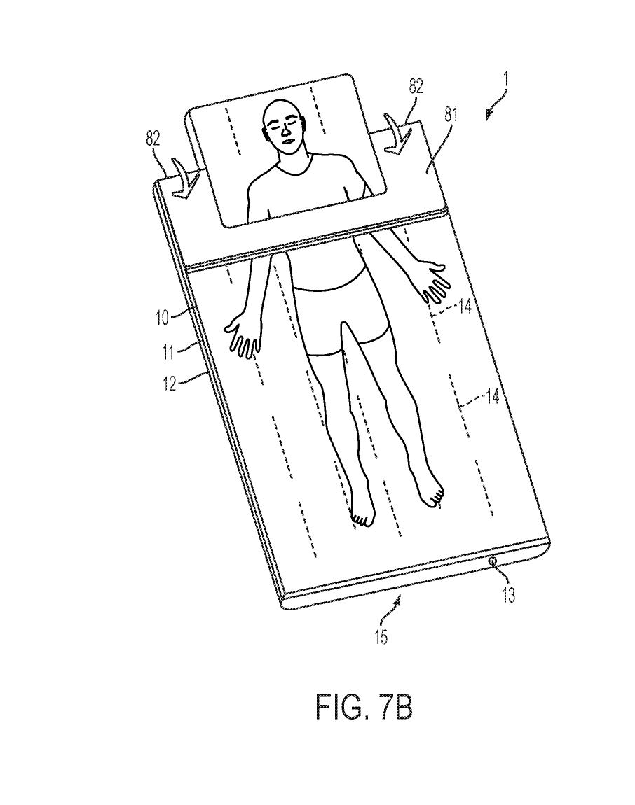

FIGS. 6, 7A, and 7B demonstrate another embodiment of the temperature regulating pad 1 having a chest warming feature. The pad 1 includes a slot 80 provided through the three layers of the pad 1, namely the air-permeable top layer 10, a perforated middle layer 11, and the bottom layer 12. In some embodiments, the slot 80 is generally U-shaped or C-shaped although other shapes can be implemented provided the structure and functionality of the resultant construction remains intact. In some embodiments, the slot 80 is integrated into the construction of the pad and the internal edges of the pad delimiting the side walls of the slot 80 are sealed and otherwise constructed in accordance with and without departing from the general structure of the pad 1 as described elsewhere herein.

With reference to FIGS. 7A and 7B, with the slot 80 positioned substantially toward one end of the pad 1, the slot 80 delimits a portion of the pad 1 shown as upper flap 81. This flap is initially located at or about the head and shoulder area of a target patient resting on the pad 1. The flap 81 will comprise substantially the same geometry as the slot 80. For example, in FIG. 7A the slot 80 is a substantially squared off U-shape. As will be apparent, the shape and size of the slot 80 is designed to conform to the relative anatomy of the patient so that the flap 81 can move over and around the patient while also leaving sufficient surface area of the pad 1 to accommodate the patient's body. In some embodiments, the width of the slot is at least half of the entire width of the pad 1 and, in some embodiments, is greater than half of the entire width of the pad 1. In some embodiments, the width of the slot 80 is at least as wide as the perforated center section 30 of the middle layer 11 shown in FIG. 3. It is appreciated that the location of the slot 80 can be varied in order to provide a flap 81 of various sizes and locations. As such, the depicted example shows the flap 81 positioned at the top of the pad 1 to provide an upper body warming feature. However, the flap 81 could be as well position toward the bottom of the pad to provide a lower body warming feature. Accordingly, reference herein to the flap 81 being a “chest warming feature” is merely illustrative as it indicates the most likely practical application but it certainly is not the only practical application.

The slot 80 releases the structure of the pad 1 to provide a means by which the flap 81 is foldable so that the flap can be folded over a portion of the pad 1 and such that the top layer 10 of the pad 1 is brought in contact with the exposed portion of the patient for supplemental temperature control. In the case where the patient is laying on his or her back, the anterior portion of the body is exposed and without the flap 81 could only otherwise be warmed (or cooled) by indirectly airflow from the pad 1. More specifically, as shown in FIGS. 7A and 7B, in some embodiments the flap 81 is configured move from a first, un-deployed position wherein the flap 81 is substantially aligned to the rest of the top surface of the pad 1 to a second, deployed position wherein the flap 81 is rotated upward and folded over toward the center of the pad 1 such that the flap 81 (and the portion of the top layer 10 thereof) rests on the torso area of a target patient resting on the pad 1. The arrows in FIG. 7B indicate the general direction of the folding action. To facilitate the folding action, in some embodiments the pad 1 includes transverse fold lines 82 comprising seal lines extending from each of terminal ends of the slot 80 to the respective outer edges of the pad 1. The fold lines 82 delimit a point of inflection or apex where the folding will occur and will allow the flap 81 to fold over with ease and precision. In some embodiments, the fold lines 82 comprise seal lines that draw together and attach the three layers of the pad 1, like the other seal lines described herein.

In some embodiments the flap 81 is configured to rest on anterior chest and torso area of the patient in order to provide temperature control, i.e. warming or cooling, capability directly to the critical core area of the patient that would not otherwise receive direct airflow from the underlying pad 1. As noted above, the location of the slot 80 and thus the flap 81 can be varied depending on where directly warming (or cooling) is desired. Likewise, the pad 1 depicted in the figures could simply be inverted if one desires to warm the lower body. Heated (or cooled) air circulates through the pad 1, including through the flap 81 and exits through the flap 81 through the portion of the top layer 10 thereof, which top layer 10 is in contact with the patient. Accordingly, the flap 81 supplements the warming (or cooling) that is already provided by the under-body portion of the pad 1 by bringing the forced air directly over the chest and torso area of the patient.

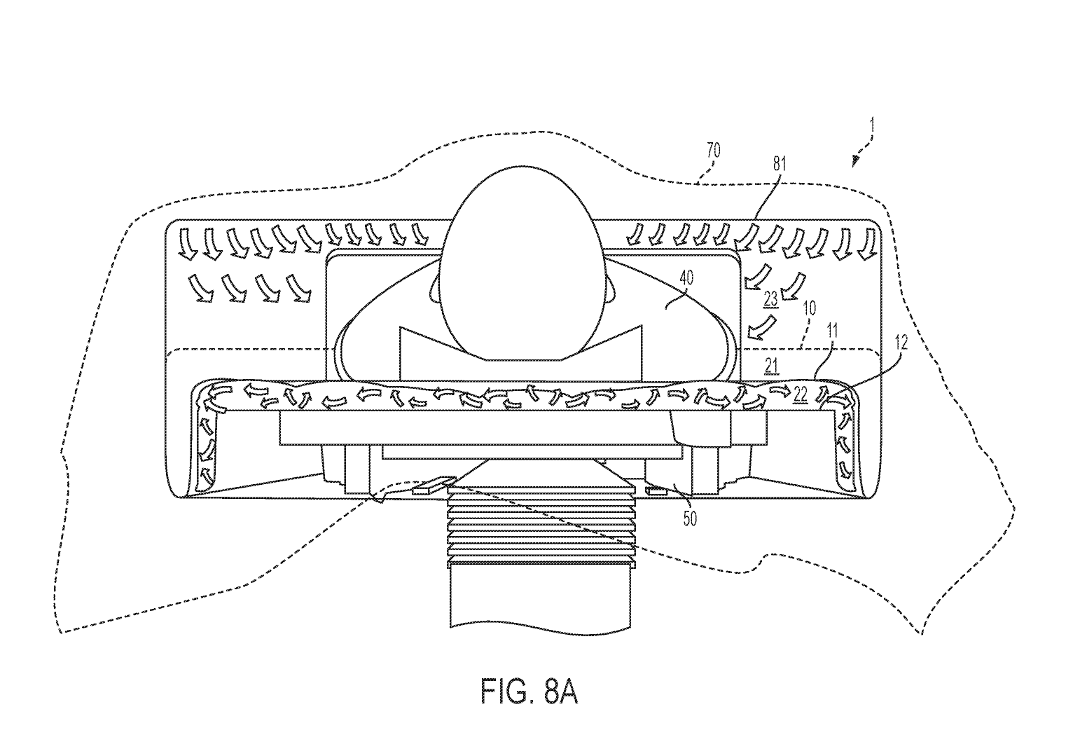

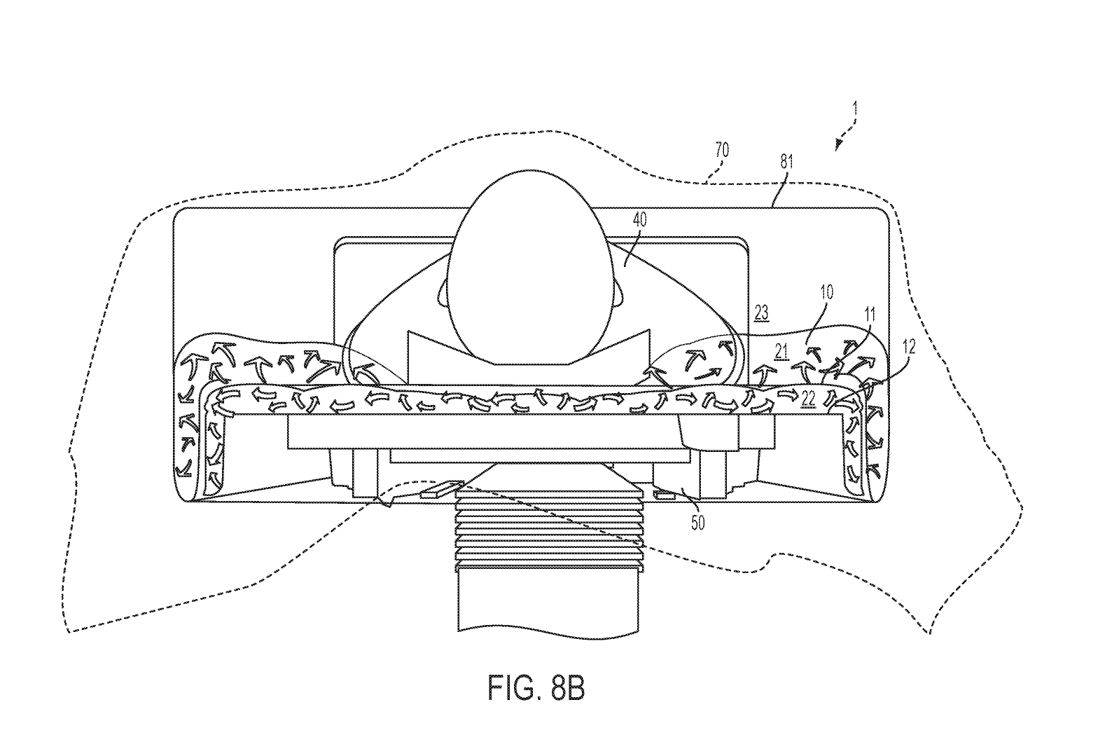

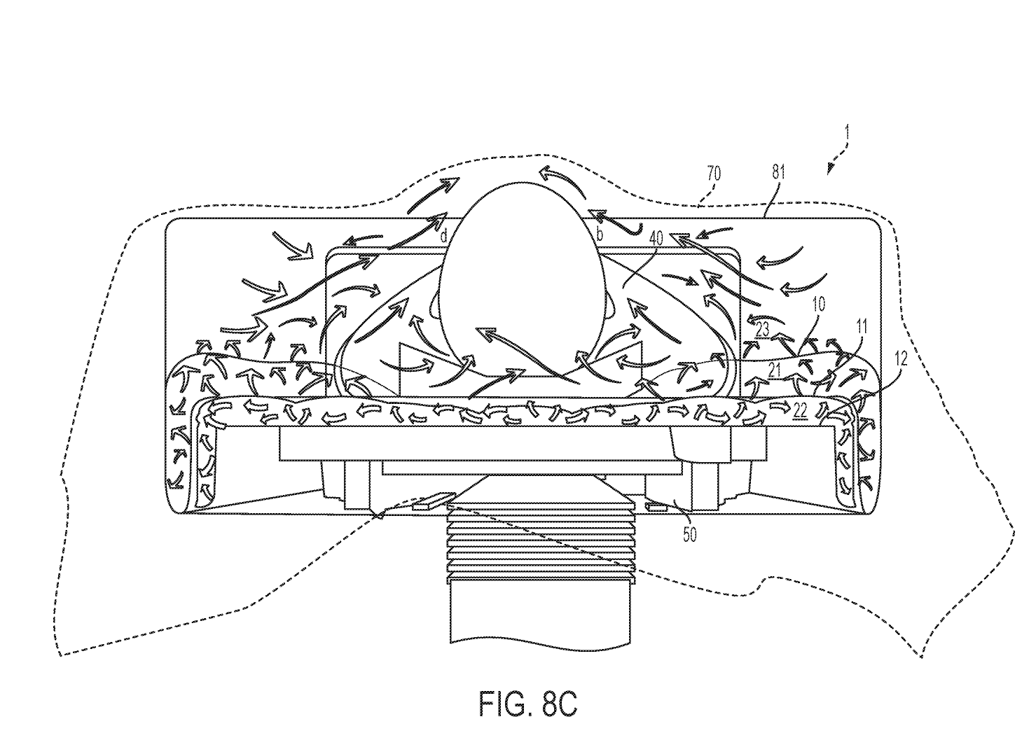

FIGS. 8A-8C show various stages of the inflated state of the temperature regulating pad 1 from an end view in use with a patient 40 resting on an underlying support surface 50. Here it can be seen more closely how flap 81 is configured to rotate upward such that it folds over, in this example, clear of the head of the patient and resting on the chest and upper torso area of the patient.

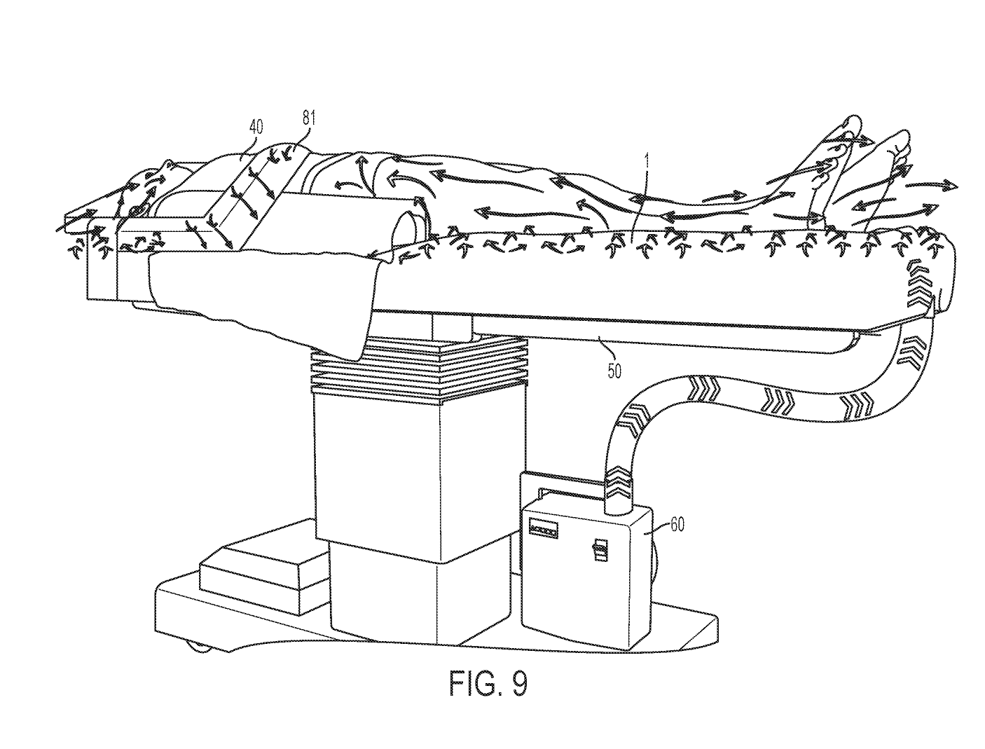

FIG. 9 shows a perspective view of the temperature regulating pad 1 in use in conjunction with a forced air source 60. Air from an external source 60 is provided into lower airspace 22 through air inlet 13 at the bottom edge of the temperature regulating pad 1. In some embodiments, air inlet 13 is configured to receive a hose or tubing from a forced air machine such as a compressor or the like that provides an air source 60. As air is forced through air inlet 13, the temperature regulating pad 1 at least partially inflates forming channels 121 disposed lengthwise in lower airspace 22 which are delimited by the plurality of seal lines 14 attaching the middle layer 11 to the bottom layer 12 (FIG. 8A). In some embodiments this causes middle layer 11 to expand upward with respect to bottom layer 12 such that the channels 121 form a substantially cylindrical or semi-cylindrical profile as shown in FIG. 5. As these channels are formed, a pressure differential is created and air proceeds from lower airspace 22 through apertures 111 of middle layer 11 and into upper airspace 21 (FIG. 8B). The air continues through the air-permeable breathable surface of the top layer 10 and into the external airspace 23 surrounding the patient 40 (FIG. 8C).

Accordingly, with a patient 40 disposed on top of top layer 10 as air is forced through the temperature regulating pad 1, air exits through the top layer to surround the patient to increase and/or maintain the patient's body temperature. Also shown is the flap 81 deployed and rest on the chest of the patient providing supplemental temperature control for the upper torso area that would not otherwise be in direct contact with the underlying pad 1. As shown in FIG. 4, air will flow through the channels 121 up and through the top layer 10 in an upward and outward direction toward the patient.

Also as shown in FIGS. 8A-8C, an optional oversheet 70 may be provided which at least partially surrounds the patient 40 and the external airspace 23. In some embodiments, the oversheet 70 may comprise a thin plastic or polypropylene material and may include adhesive portions and/or an adhesive border to secure the oversheet 70 to the sides or bottom of the underlying support surface 50.

With the air initially forced into lower airspace 22, the air pressure inside lower airspace 22 is greater than the pressure inside upper airspace 21. The air pressure inside upper airspace 21 is greater than that of external airspace 23. Thus, a pressure gradient or differential is created with higher pressure lower airspace 22 as compared to that of the external airspace 23. As the air enters inlet 13 and into lower airspace 22, channels 121 and then the air flows through the apertures 111 of the middle layer 11 and into upper airspace 21. Due to the pressure differential, the air then moves through the permeable top layer 10 into the external airspace 23. The air exiting the top layer 10 is at a much lower pressure and velocity in the air exiting from the lower airspace 22 to the upper airspace 21 through middle layer 11. Accordingly, the turbulence of the air exiting through top layer 10 is less than the turbulence of the air exiting through middle layer 11. Thus, the air exiting top layer 10 toward the patient is much less turbulent than the air exiting the middle layer 11. The temperature regulating pad 1, therefore, is effective in reducing the air turbulence around the patient thereby minimizing the production and movement of dust in the operating room while still adequately maintaining or increasing the body temperature of the patient. In that sense, any residual dust inside the pad 1 from manufacturing or storage is less likely to be pushed through and out of the pad 1 and into the surgical environment. In some embodiments, therefore, the top layer 10 is air-permeable to function as an air filter to permit the flow of air out but prevent or significantly limit the flow of dust and other particulates.

FIG. 9 shows an embodiment of the temperature regulating pad 1 showing the various airspaces with an exemplary patient 40 disposed thereon and subjected to the air flow that the pad 1 herein provides, including the flap 81 which provides temperature regulation to the chest area.

The temperature of air provided through the pad 1 can vary as needed although in many instances relatively warm air will be desired in order to maintain the patient's body temperature during a procedure such as surgery. In some embodiments, the forced air is relatively warm, between 22 and 43 degrees Celsius, in order to regulate the patient's body temperature toward the desired 36-37 degrees Celsius range and to otherwise prevent or alleviate hypothermia. In other embodiments, the forced air may be relatively cool, between 18 and 22 degrees Celsius, in order to reduce the patient's body temperature in the case of hyperthermia, pyrexia (fever), or other scenarios where it is desirous to reduce the patient's body temperature.

As noted above, certain materials may be selected for each of the top layer 10, middle layer 11, and bottom layer 12 to promote the functionality of the pad 1. For example, the top layer 10 may comprise a comfortable material such as polypropylene that is air permeable and breathable to permit air to pass through but prevents or limits the passage of dust or other particulates. In some embodiments, the top layer 10 is permeable to air but prevents or substantially reduces the passage of dust or other particulate, in effect acting as an air filter. The middle layer in some embodiments comprises a thin plastic that includes the aforementioned apertures 111 and also permits for seal lines to be created between it and the bottom layer 12, which seal lines 14 may be constructed by heat sealing or other known construction methodology. In some embodiments bottom layer 12 comprises an impermeable low friction material such as nylon to facilitate movement of a patient with respect to an underlying support surface. It is appreciated and understood that the temperature regulating pad 1 is generally suitable for regulating the temperature of a patient and need not necessarily be used with warm air. In certain applications, the pad 1 could be equally used with forced cool air and would function substantially as explained and described herein.

The temperature regulating pad 1, in some embodiments comprises an air-permeable polypropylene top layer 10, a perforated plastic middle layer 11, and an impermeable low friction nylon bottom layer 12. The perforated middle layer 11 is disposed between the top layer 10 and the bottom layer 12 and is at least partially attached to the bottom layer by a plurality of spaced apart seal lines 14. The top layer 10 and the perforated middle layer 11 delimit an upper airspace 21. The perforated middle layer 11 and the bottom layer 12 delimit a lower airspace 22. Forced air of a desired temperature is received into the lower airspace 22 and flows from the lower airspace 22 into the upper airspace 21 through the perforated middle layer 11. The forced air exits the air-permeable top layer 10 into an external airspace 23 about said top layer 10 to regulate the temperature of a patient 40 resting on the pad. The air exiting the air-permeable top layer 10 into the external airspace 23 is less turbulent than the air flowing from the lower airspace 22 to the upper airspace 21 to limit the excitement of dust and particulate in and around the area surrounding the pad.

While specific embodiments have been described in detail in the foregoing detailed description and illustrated in the accompanying drawings, those with ordinary skill in the art will appreciate that various modifications and alternatives to those details could be developed in light of the overall teaching of the disclosure. Accordingly, the particular arrangements disclosed are meant to be illustrative only and not limiting as to the scope of the invention, which is to be given the full breadth of the appended claims in any and all equivalents thereof

What is claimed is:

1. A temperature regulating pad, comprising:
an air-permeable top layer, a perforated middle layer, and a bottom layer, said perforated middle layer disposed between said top layer and said bottom layer;
said top layer and said perforated middle layer delimiting an upper airspace and said perforated middle layer and said bottom layer delimiting a lower airspace;
a slot disposed through each of said top layer, said middle layer, and said bottom layer, said slot delimiting a foldable flap, said flap configured to move between at least a first position and a second position; and
wherein forced air is received into said lower airspace and flows from said lower airspace into said upper airspace through said perforated middle layer, exiting said air-permeable top layer into an external airspace about said top layer.

2. The temperature regulating pad of claim 1, wherein in the first position said flap is substantially flat and parallel with said pad and wherein in the second position said flap is folded over a portion of said pad.

3. The temperature regulating pad of claim 1, wherein said slot is substantially U-shaped.

4. The temperature regulating pad of claim 1, wherein said slot has first and second terminal ends, and a transverse fold line extends from each of said first and second terminal ends to a respective edge of said pad.

5. The temperature regulating pad of claim 1, wherein said air exiting said air-permeable top layer into said external airspace is less turbulent than said air flowing from said lower airspace to said upper airspace.

6. The temperature regulating pad of claim 1, wherein said perforated middle layer is at least partially attached to said bottom layer.

7. The temperature regulating pad of claim 3, wherein said perforated middle layer is at least partially attached to said bottom layer by a plurality of spaced apart seal lines.

8. The temperature regulating pad of claim 1, wherein said plurality of spaced apart seal lines delimit one or more longitudinal channels within said lower airspace.

9. The temperature regulating pad of claim 1, further comprising an inlet in flow communication with said lower airspace, wherein said inlet receives said forced air.

10. The temperature regulating pad of claim 1, wherein said perforated middle layer includes a perforated center section surrounded by an impermeable border.

11. The temperature regulating pad of claim 1, wherein said bottom layer comprises a low friction nylon material.

12. The temperature regulating pad of claim 1, wherein said middle layer comprises plastic.

13. The temperature regulating pad of claim 1, wherein said top layer comprises polypropylene.

14. A temperature regulating pad, comprising:
an air-permeable top layer, a perforated middle layer, and a bottom layer, said perforated middle layer disposed between said top layer and said bottom layer;
said top layer and said perforated middle layer delimiting an upper airspace and said perforated middle layer and said bottom layer delimiting a lower airspace;
a slot disposed through each of said top layer, said middle layer, and said bottom layer, said slot delimiting a foldable flap configured to move between first position and a second position;
wherein forced air is received into said lower airspace and flows from said lower airspace into said upper airspace through said perforated middle layer, exiting said air-permeable top layer into an external airspace about said top layer; and
wherein said air exiting said air-permeable top layer into said external airspace is less turbulent than said air flowing from said lower airspace to said upper airspace.

15. The temperature regulating pad of claim 11, wherein said perforated middle layer is at least partially attached to said bottom layer.

16. The temperature regulating pad of claim 12, wherein said perforated middle layer is at least partially attached to said bottom layer by a plurality of spaced apart seal lines.

17. The temperature regulating pad of claim 11, wherein said plurality of spaced apart seal lines delimit one or more longitudinal channels within said lower airspace.

18. The temperature regulating pad of claim 11, further comprising an inlet in flow communication with said lower airspace, wherein said inlet receives said forced air.

19. The temperature regulating pad of claim 11, wherein said perforated middle layer includes a perforated center section surrounded by an impermeable border.

20. The temperature regulation pad of claim 11, further including an oversheet configured to surround said external airspace.
